(19) United States
(12) Patent Application Publication
(10) Pub. No.: US 2019/0369595 A1
(43) Pub. Date: Dec. 5, 2019

(54) SYSTEM, METHOD AND COMPUTER PROGRAM PRODUCT FOR DETERMINING A NUISANCE GENERATED BY AN INDUSTRIAL INSTALLATION, AND INDUSTRIAL INSTALLATION EQUIPPED WITH THE SYSTEM

(71) Applicant: SUEZ INTERNATIONAL, PARIS LA DEFENSE CEDEX (FR)

(72) Inventors: Eric FIEVEZ, SURESNES (FR); Mathieu DELAHAYE, JOUY EN JOSAS (FR); Valérie NASTASI, VILLEJUST (FR)

(21) Appl. No.: 16/472,699

(22) PCT Filed: Dec. 22, 2017
(86) PCT No.: PCT/EP2017/084507

§ 371 Date: Jun. 21, 2019

(30) Foreign Application Priority Data
Dec. 22, 2016 (FR) 1663178

(51) Int. Cl. G05B 19/406; G01P 5/02; G01H 3/00; G01N 33/00; B64C 39/02

(52) U.S. Cl. CPC G05B 19/406; G01P 5/02; G01H 3/00; G05B 2219/31449; B64C 39/024; B64C 2201/126; G01N 33/0062

(57) ABSTRACT

Disclosed is a system for determining a level of nuisance generated by an industrial installation, including at least one aerial drone, the aerial drone being controllable to move to a measurement point above the industrial installation, and to determine, at the measurement point, by way of at least one sensor fitted to the aerial drone, at least one level of a quantity generating the nuisance, the system furthermore including a treatment unit configured to determine the nuisance level on the basis of the level of the sensed quantity

### TECHNICAL FIELD

The invention relates to a system and a method for determining a level of nuisance generated by an industrial installation.

The invention also relates to a computer program product for implementing the method according to the invention and an industrial installation equipped with the system according to the invention.

### PRIOR ART

Sewage or waste treatment plants or other industrial installations in other fields are the source of nuisances, notably olfactory or sound nuisances, in particular affecting local residents, and sources of emission of greenhouse gases, pollutant and/or hazardous (explosive) gases.

In order to provide a set of measurements sufficient for effectively managing the odor (or sound) emissions of an industrial installation, the plant constructors or operators use:
either stationary sensors that need to be installed in large numbers in order to be able to collect sufficient data, or to have sufficient information in real time, or to be able to supply input for their odor dispersion prediction software. However, these sensors are installed close to the ground (rarely above 2 m) and in fixed locations. The cost of installation and maintenance is multiplied by the number of sensors;
or spot measurements made by mobile sensors. This method requires the presence of staff and is only possible intermittently, during the performance testing of a new installation, for example.

One object of the invention is to provide a system for determining a level of nuisance generated by an industrial installation that reduces the number of collecting devices needed for capturing this data.

One object of the invention is to provide a system for determining a level of nuisance generated by an industrial installation, whereof the cost of installation and maintenance is less than the systems known to the prior art.

### Disclosure of the Technique

At least one object of the invention is achieved with, according to a first aspect of the invention, a system for determining a level of nuisance generated by an industrial installation.

The system according to the invention comprises at least one aerial drone, said aerial drone being controllable for moving to a measuring point above, or near, the industrial installation, and for determining, at the measuring point at least one level of a quantity generating said nuisance, by means of at least one sensor fitted to said aerial drone.

Quantity, in the context of the present invention, is understood to mean a physical quantity and/or a chemical quantity and/or a biological quantity.

Near the industrial installation is understood to mean within a radius of about 5 km.

The system according to the invention further comprises a processing unit configured for determining the level of nuisance based on the level of the sensed quantity.

The implementation of an aerial drone makes it possible to reduce the number of sensors to be installed.

Furthermore, the system makes it possible to measure the nuisances over the whole of a site, or even at a location predetermined in advance.

The aerial drone may be intermittently moved to a measuring point at which it is not possible to install a fixed sensor, or at least at which installing a fixed sensor would cause high maintenance costs due, notably, to the necessary short-term replacement of said fixed sensor. Furthermore, the aerial drone after determining a level of nuisance at a first measuring point may move to a second measuring point in order to determine a level of nuisance at the second measuring point. Thus, the system of determination according to the invention makes it possible to minimize the investment in the number of sensors, and thus the cost of maintaining the sensors.

By industrial installation, the invention refers, for example, to municipal and industrial water treatment and waste plants, such as waste landfill centers, sorting centers, incinerators, composting plants, which are sources of olfactory and/or sound pollution, pollutant gases, greenhouse gases and/or hazardous sources. Any other industrial installation source of olfactory and/or sound nuisances is potentially concerned.

Nuisance generated, is thus understood to mean, for example, an olfactory and/or sound pollution, the presence of pollutant gases, a source of greenhouse gases, or the presence of hazardous elements.

The system comprises at least one aerial drone, i.e. one or more aerial drones.

The aerial drone may use intrinsic or extrinsic energy as part of an external power supply.

The aerial drone may be remotely controlled by means of a wired or wireless connection.

The processing unit may be fitted in the industrial installation or be located remotely from said industrial installation or be embedded on the aerial drone.

The aerial drone may comprise multiple sensors, optionally of different types. For example, an aerial drone may comprise multiple sensors of different sizes.

The following sensors are envisaged, but not limited to: ammonia (NH3) sensor, mercaptan (RSH) sensor, H2S sensor, volatile organic compounds (VOCs) sensor, noise sensor (sound level meter), anemometer, electronic noses (electronic noses are systems that detect and measure odorous chemical compounds and by processing the data make it possible to quantify the level of odor), multisensors making it possible to determine the concentrations in odorous pollutants and to calculate the concentration in units of odor, and other types, such as NO2, SO2, CO, NOx.

The system according to the invention may comprise wired or wireless means for transmitting the level of the sensed quantity, to the processing unit.

The system according to the invention may comprise means for regulating the nuisance generated by the industrial installation, servo-controlled according to the determined level of nuisance. The regulating means may, for example, be a servo-controlled solenoid valve.

The at least one sensor may be an anemometer. Knowledge of the direction, sense and intensity of the wind at the measuring point may help refine predictions relating to the flow transport of the nuisance generated by the industrial installation.

The at least one sensor is a sound sensor, typically a microphone or sound level meter. The system according to the invention may then be used to determine a sound nuisance level.

The at least one sensor may be a gas concentration sensor. The system according to the invention may then be used to determine an olfactory nuisance level.

The at least one aerial drone may be fitted with a boom arranged for having the sensor at a distance from said aerial drone. This boom may then be used to minimize the influence of the presence of the aerial drone on the determined level of the quantity. This boom may be useful when it is desired to reduce the influence of the rotation of the propellers of the aerial drone on a wind measurement performed by an anemometer, on a sound measurement performed by a sound sensor, on a temperature measurement performed by a temperature sensor, a thermometer.

Preferably, the system according to the invention comprises wireless communication means fitted in the aerial drone and the industrial installation and which are configured for establishing wireless communication between the aerial drone and the industrial installation.

According to one embodiment, the aerial drone comprises a transmission unit configured for receiving movement commands. The transmission unit may be configured for transmitting the level of the sensed quantity to the processing unit.

Advantageously, movement commands to the controllable aerial drone and the level of the quantity may be transmitted according to a wireless protocol, e.g. the WiFi protocol.

The system according to the invention may comprise wired or wireless communications relays for transmitting movement commands to the aerial drone and/or the level of the quantity.

According to a second aspect of the invention a method is provided for determining a level of nuisance generated by an industrial installation, implemented in a system of determination according to the first aspect of the invention, or one or more of its improvements, comprising the steps of:
control of at least one aerial drone equipped with at least one sensor of a quantity generating said at least one nuisance, along a trajectory leading it to a measuring point,
determination, by said sensor, of a level of said quantity at the measuring point,
determination, by a processing unit of the system, of said level of nuisance based on said value of said quantity.

Multiple determinations by the at least one sensor may be performed during the same flight of the at least one aerial drone.

Multiple commands may be performed during the same flight of the at least one aerial drone.

The measuring point may be located at the property boundary of the industrial installation. By property boundary, the present description refers to any point located on the border, both horizontal and vertical, separating the property comprising the industrial installation from the outside of said property.

According to a feature of the method according to the invention, the steps of control and/or determination may be performed at regular intervals.

Preferably, the trajectory of the at least one aerial drone may fall within a predetermined overflight zone of the industrial installation. The overflight zone may be determined from regulations that apply to the industrial installation level. The overflight zone may be determined so as to limit the exposure of the staff of the industrial zone to the circulation of the at least one aerial drone.

Advantageously, the method according to the invention comprises a step of regulating the nuisance generated by the industrial installation based on the determined level of nuisance. For example, a valve may be operated by an operator based on knowing the determined level of nuisance. When a means of regulation comprises a means of control of said means of regulation, e.g. when it is a solenoid valve, the step of regulating may comprise a servo-control of the means of regulation.

The method according to the invention may comprise a step of predicting the spatial evolution of the nuisance, and more particularly the intensity of the nuisance, based on a model receiving the determined level of nuisance as input.

The step of predicting the spatial evolution may comprise a recalibration of the model by the determined level of nuisance. The determination of a level of nuisance may thus help improve the prediction of the spatial evolution of the nuisance.

According to another aspect of the invention, a computer program product is provided, downloadable from a communication network and/or stored on a computer-readable medium and/or executable by a microprocessor, characterized in that it comprises program code instructions for the implementation of a method for determining a level of nuisance generated by an industrial installation according to the first aspect of the invention, or one or more of its improvements.

According to yet another aspect of the invention an industrial installation is provided equipped with a system of determination according to the second aspect of the invention, or one or more of its improvements.

The industrial installation may be equipped with a processing unit configured for determining the level of nuisance based on the level of the sensed quantity.

### DESCRIPTION OF THE FIGURES

Other features and advantages of the invention will become apparent on reading the detailed description of implementation and embodiment in no way restrictive, with respect to the appended figures in which:

FIG. 1 illustrates a first embodiment of a system according to the invention.

### DESCRIPTION OF THE INVENTION

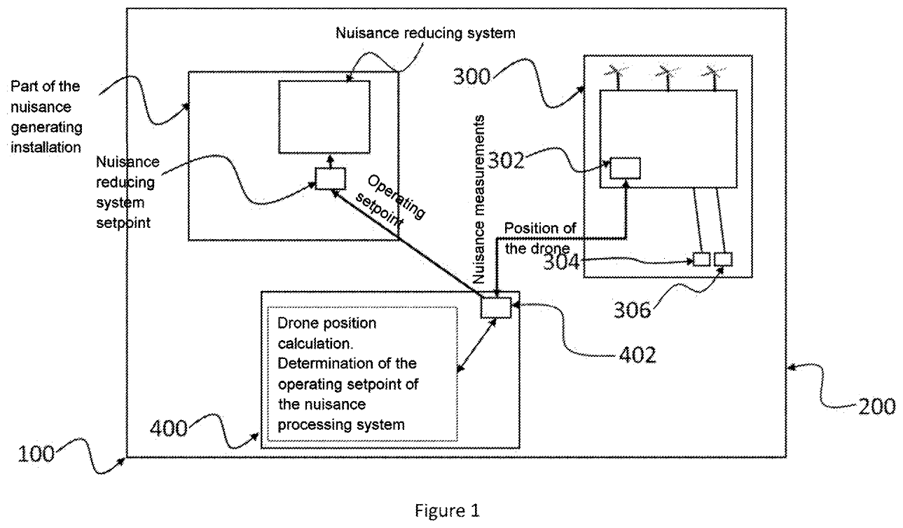

Since this embodiment is in no way restrictive, variants of the invention may notably be produced comprising only a selection of features subsequently described, as described or generalized, isolated from the other features described, if this selection of features is sufficient to impart a technical advantage or to differentiate the invention from the prior art. With reference to FIG. 1, an embodiment is illustrated of a system 100 for determining a level of nuisance generated by an industrial installation 200. In the illustrated example, the nuisance is olfactory.

The system 100 comprises:
an aerial drone 300,
a processing unit 400 fitted in the industrial installation 200,
a transmission unit 402 fitted in the industrial installation 200.

The aerial drone 300 comprises a transmission unit 302 configured for receiving movement commands, from the transmission unit 402. The aerial drone 300 is controllable for moving to a measuring point above, or near, the industrial installation 200.

The aerial drone 300 comprises two sensors 304 and 306, respectively an anemometer 304 and a gas concentration sensor 306. The aerial drone 300 is configured for determining, at the measuring point, a level of gas concentration generating the nuisance, olfactory in the illustrated example.

The transmission unit 302 is further configured for transmitting the level of the sensed quantity to the processing unit, via the transmission unit 302, as well as the direction, sense and intensity of the wind sensed by the anemometer 304. This data is received by the processing unit 400 via the transmission unit 402.

The data collected in flight is transferred, wholly or partly during the flight, via radio communication or WiFi or any other method of wireless communication.

The aerial drone 300 may be controlled to move to different strategic locations of the industrial installation, e.g.:
at the main sources of emissions of nuisances (e.g. at the sludge storage in a WWTP, at the entrance to the pretreatment premises, at the sludge thickener, at the outlet flue of the deodorization system, etc.),
at the property boundary,
at nearby housing areas,
at altitude in order to measure the future of the olfactory molecules once diffused.

The aerial drone 300 may be controlled to have multiple route paths in order, for example, to pass through locations with high concentrations of gas then through locations with a low concentration of pollutants.

The aerial drone 300 may be controlled for being positioned hovering at different points of the route for the time necessary for a stable and reliable measurement of the different sensors, for a duration ranging from a second to a minute, for example.

To improve the quality of the measurement, the aerial drone 300 is fitted with a boom arranged for having the sensors 304 and 306 at a distance from said aerial drone.

Once its route is completed the aerial drone returns to a base (not represented) for the purpose of recharging its battery (not represented) in order to carry out the next controlled route,

The processing unit 400 is configured for determining the level of nuisance based on the level of said sensed quantity.

Based on the sensed quantity, an operator may take preventive or corrective actions to minimize the olfactory impact of the industrial installation. These actions may, for example, be the following:
bringing forward odor maintenance operations,
optimizing odor treatment on the site (e.g. by adapting the reagent levels, modifying the inflows into the deodorization),
modifying the ventilation systems,
starting up an odor dispersal system,
closing the premises,
bringing forward the intervention of service providers,
modifying the operation of a process of the industrial installation.

When the industrial installation is equipped with an odor monitoring tool that uses the data received from the aerial drone for modeling and simulating the concentrations of odor molecules over time, these actions may be automated via the odor monitoring tool. The odor monitoring prediction tool may comprise a recalibration of the model by the determined level of nuisance.

The route of the aerial drone may be automatically defined based on the mapping of the odors determined by the monitoring tool.

For example, when the pollutant concentration at the outlet of a deodorization tower flue is greater than a setpoint, the flow of air entering deodorization (or the reagent treatment level) may be automatically increased. Thus, the industrial installation may be equipped with means for regulating the nuisance generated, servo-controlled according to the determined level of nuisance.

According to an additional use, the system according to the invention may be implemented for the purpose of solving the operating problems of the industrial installation.

The aerial drone may, for example, be sent to critical locations where there is a suspected malfunction, e.g. a pumping station or a leak in a building.

The aerial drone may be controlled to be positioned for a few minutes at the outlet of the flue from the deodorizing towers in order to measure whether they are operating correctly and to identify a possible malfunction. According to the collected data, the operator may take the necessary corrective measures.

This is difficult to achieve with fixed sensors since the outlet of the flue is a location that is difficult to access. Their installation would subject them to difficult conditions (humidity, for example) reducing their service life and reliability.

According to yet another use, the system according to the invention may be implemented for monitoring treatment guarantees during the construction of the industrial installation. Indeed, the constructor of an industrial installation must comply with guarantees at the property boundary in terms of odor concentration and noise level. This involves differences in values between the initial level (without the industrial installation) and the level found during the operation of the industrial installation. It is then necessary to have a good knowledge of the initial level for clearly distinguishing the odors or noise originating from the new industrial installation from those originating from the existing environment. The invention makes it possible to make a complete initial map by flying over and taking a large number of measurements on the site for mapping the area which is not achievable with fixed sensors. This same diagnosis is then made after the construction of the industrial installation.

Of course, the invention is not limited to the examples which have just been described and numerous adjustments may be made to these examples without departing from the scope of the invention. In addition, the various features, forms, variants and embodiments of the invention may be combined with each other in various combinations insofar as they are not incompatible or mutually exclusive of each other.

1. A system for determining (100) a nuisance level generated by an industrial installation (200), comprising at least one aerial drone (300), said aerial drone being controllable for moving to a measuring point above or near said industrial installation, and for determining, at the measuring point, at least one level of a quantity generating said nuisance, by means of at least one sensor (304, 306) fitted to said aerial drone, said system further comprising a processing unit (400) configured for determining said level of nuisance based on said level of said sensed quantity.

2. The system as claimed in claim 1, further comprising means for regulating the nuisance generated by the industrial installation, servo-controlled according to the determined level of nuisance.

3. The system as claimed in claim 1, wherein the at least one sensor is an anemometer (304).

4. The system as claimed in claim 1, wherein the at least one sensor is a sound sensor.

5. The system as claimed in claim 1, wherein the at least one sensor is a gas concentration sensor (306).

6. The system as claimed in claim 1, wherein the aerial drone is fitted with a boom arranged for having the sensor at a distance from said aerial drone.

7. The system as claimed in claim 1, further comprising wireless communication means fitted in the aerial drone and the industrial installation and configured for establishing wireless communication between the aerial drone and the industrial installation.

8. A method for determining a level of nuisance generated by an industrial installation, implemented in a system of determination as claimed in claim 1, comprising the steps of:
control of at least one aerial drone equipped with at least one sensor of a quantity generating said at least one nuisance, along a trajectory leading it to a measuring point,
determination, by said sensor, of a level of said quantity at the measuring point,
determination, by a processing unit of the system, of said level of nuisance based on said value of said quantity.

9. The method as claimed in claim 8, wherein the measuring point is located at the property boundary of the industrial installation.

10. The method as claimed in claim 8, wherein the steps of control and determination are performed at regular intervals.

11. The method as claimed in claim 8, wherein the trajectory falls within a predetermined overflight zone of the industrial installation.

12. The method as claimed in claim 8, further comprising a step of regulating the nuisance generated by the industrial installation based on the determined level of nuisance.

13. The method as claimed in claim 8, comprising a step of predicting the spatial evolution of the nuisance based on a model receiving the determined level of nuisance as input.

14. The method as claimed in claim 8, wherein the step of predicting the spatial evolution comprises a recalibration of the model by the determined level of nuisance.

15. A non-transitory computer-readable medium on which is stored a computer program which, when executed by a microprocessor, causes the microprocessor to perform the method for determining a level of nuisance generated by an industrial installation as claimed in claim 8.

16. An industrial installation equipped with a system of determination as claimed in claim 1.

17. The system as claimed in claim 2, wherein the at least one sensor is an anemometer (304).

18. The system as claimed in claim 2, wherein the at least one sensor is a sound sensor.

19. The system as claimed in claim 3, wherein the at least one sensor is a sound sensor.

20. The system as claimed in claim 2, wherein the at least one sensor is a gas concentration sensor (306).
